(19) United States
(12) Patent Application Publication
(10) Pub. No.: US 2010/0172840 A1
(43) Pub. Date: Jul. 8, 2010

(54) PREPARATION OF CARBON NANOTUBES WITH LANTHANOID CATALYSTS

(75) Inventors: Balaji SITHARAMAN, Lake Grove, NY (US); Magdalena Swierczewska, Mount Sinai, NY (US)

Correspondence Address: KENYON & KENYON LLP, ONE BROADWAY, NEW YORK, NY, 10004, US

(73) Assignee: The Research Foundation of State University of New York, Albany, NY (US)

(21) Appl. No.: 12/569,749

(22) Filed: Sep. 29, 2009

Related U.S. Application Data
(60) Provisional application No. 61/100,862, filed on Sep. 29, 2008.

(51) Int. Cl. A61K 49/00; C01F 17/00; B01J 23/10; B01J 21/06; D01F 9/12

(52) U.S. Cl. 424/9.1; 423/263; 502/302; 502/263; 423/447.3; 423/447.2; 977/742; 977/773; 977/775; 977/777; 977/842; 977/843; 977/750

(57) ABSTRACT

A lanthanoid metal catalyst for the formation carbon nanotubes from a carbon-containing gas mixture, a method for the formation of carbon nanotubes with the lanthanoid metal catalyst, endohedral carbon nanotube complexes containing lanthanoid metal atoms and/or ions, carbon nanotube imaging contrast agents, and a method for imaging living tissue with carbon nanotube imaging contrast agents are provided.

### RELATED APPLICATIONS

This application claims benefit to U.S. Provisional Patent Application No. 61/100,862, filed Sep. 29, 2008, the disclosures of which are incorporated herein by reference.

### FIELD OF THE INVENTION

The present invention relates to nanotechnology and carbon nanotubes. In particular, the present invention is directed to a process for the production of single-wall carbon nanotubes (SWNTs) and endohedral SWNT-metal complexes, catalysts for use in the process of the invention, and carbon nanotube-based contrast agents for noninvasive imaging.

### BACKGROUND

Single-walled carbon nanotubes (SWNTs) possess a number of interesting and unique physio-chemical properties, making SWNTs uniquely suitable for a wide range of applications in the material and biomedical sciences. A variety of techniques have been developed for the synthesis of SWNTs, including arc discharge (Z. Shi et al., Carbon, 1999, 37:1449-1453), laser ablation (Y. Zhang and S. Iijima, Appl. Phys. Lett., 1999, 75:3087), and chemical vapor deposition (CVD) (M. Su, B. Zheng and J. Liu, Chem. Phys. Lett., 2000, 322:321-326).

In the CVD process, metal nanoparticles act as catalysts for carbon feedstock cracking and for the nucleation and growth of SWNTs. The structure, length, and yield of carbon nanotubes (CNTs) are affected not only by CVD-related parameters, such as the feedstock, pressure, growth time, and temperature, but also by the composition and size of the metal catalysts. The transition metals iron, cobalt, and nickel have been the most widely used catalysts for the CVD synthesis of SWNTs synthesis, and have been generally accepted in the art as the most suitable catalysts, based on metal-carbon binary phase diagrams (C. P. Deck and K. Vecchio, Carbon, 2006, 44:67-275).

Other transition metals and alloys have been investigated as catalysts to provide insight into the growth mechanisms of SWNTs, and to provide a degree of control over the various properties of SWNTs. For example, the use of gold (S. Bhaviripudi et al., J. Am. Chem. Soc., 2007, 129:1516-1517), tungsten (C. Jin Lee et al., Chem. Phys. Lett., 2002, 361:469-472), molybdenum (Y. Li, J. Liu, Y. Wang and Z. L. Wang, Chem. Mater., 2001, 13:1008-1014), copper (Cu), platinum (Pt), palladium (Pd), manganese (Mn), chromium (Cr), tin (Sn), magnesium (Mg), and aluminum (Al) have all been demonstrated to catalyze SWNT formation (D. Yuan et al., Nano Lett., 2008, 8:2576-2579). The use of bimetallic catalysts, such as Co/Ni and Rh/Pd has also been reported (X. P. Tang et al., Science, 2000, 288:492; see also U.S. Patent Application Publication No. US 2008/0095695).

Investigations of the f-block transition elements, also known as the inner-transition elements, as catalysts for CNT and/or SWNT synthesis have been limited. The inner-transition elements are not known to be as highly catalytic as the transition elements. For example, as set forth in the prior art, lanthanoids, such as gadolinium, europium, and terbium, are characterized by low carbon solubility, slow carbon diffusion, and limited carbide formation, any of which would make the lanthanoids unacceptable as catalysts for SWNT nucleation and growth (C. P. Deck and K. Vecchio, Carbon, 2006, 44:267-275).

The control of the many variable properties of CNTs, such as the multiplicity of walls and the pitch, diameter, length, conductivity, and uniformity of the tubes, is far from satisfactory. Other variables, such as yield, throughput, cost, and catalyst lifetime, are also imperfectly controlled. Despite the considerable effort that has been expended in this field by a number of scientific and engineering teams, there remains a need for selective and efficient methods of CNT and SWNT production.

In addition, the advent of numerous noninvasive imaging modalities, such as X-ray, computed tomography, single photon-emission-computed tomography, positron emission tomography (PET), magnetic resonance imaging (MRI), ultrasound imaging, radio frequency (rf), and optical imaging allows scientists and clinicians to acquire in vivo images of the anatomy and physiology of animals and humans. Each of the in vivo imaging techniques has characteristic strengths and weaknesses. For each imaging modality, substantial attention has been devoted to developing contrast agents not only for improving the contrast of the acquired images, but also for molecular imaging targeting specific biomolecules, cell tracking, and gene expression. Hybrid imaging modalities, such as thermo-acoustic (TA) tomography (TAT) and photo-acoustic (PA) tomography (PAT), have been developed for different applications, and, when combined, those two imaging modalities provide a single imaging system for early breast cancer detection.

Diagnosis of cancer in its early stages depends on the recognition of subtle changes in tissue properties, such as mechanical properties, optical absorption, and rf absorption. For example, changes in ion and water concentrations lead to changes in rf absorption. To detect these changes, non-ionizing rf electromagnetic waves and visible/near infrared (NIR) light-based imaging modalities have generated particular interest over the last decade.

TAT/PAT synergizes the advantages of pure ultrasound and pure rf/optical imaging, allowing both satisfactory spatial resolution and high soft-tissue contrast. For instance, PAT is a unique noninvasive technology for imaging and quantifying the levels of vascularization and oxygen saturation in tumors. These features are associated with angiogenesis and hypoxia accompanying malignant tumors.

TAT/PAT is also capable of revealing information such as water/ion concentration, blood volume, and oxygenation of hemoglobin. Because these parameters can change during the early stages of cancer, TAT/PAT offers opportunities for early detection. However, even though high rf and optical contrast exists between well-developed malignant tumor tissue and normal human breast tissue, the contrast during very early stages of cancer has not always been sufficient. Thus, a targeted contrast agent could be greatly beneficial for early cancer diagnosis using TAT/PAT.

### SUMMARY OF THE INVENTION

The present invention is directed to inner-transition elements as selective catalysts for CVD-based SWNT synthesis. The synthesis of SWNTs is preferably carried out by CVD of carbon feedstock on nanoparticulate Gd, Eu, and Tb catalysts prepared by a self-organizing block copolymer templating technique. The SWNTs produced by the method take the form of endohedral complexes, with Gd, Eu, and Tb atoms within the tube interiors.

The present invention thus provides a catalyst for the formation carbon nanotubes from a carbon-containing gas mixture, where the catalyst comprises nanoclusters of at least one lanthanoid metal. Preferably, the lanthanoid is gadolinium, europium, terbium, or a mixture of at least two of gadolinium, europium, and terbium. The nanoclusters are preferably disposed on the surface of a refractory support material, where the support material is preferably selected from the group consisting of mica, quartz, and silicon, and is more preferably silicon.

The present invention also provides a method for the formation of carbon nanotubes. The method comprises providing a catalyst comprising nanoclusters of at least one lanthanoid metal, contacting a carbon-rich gas with the catalyst, and forming carbon nanotubes. The carbon nanotubes are preferably single-wall carbon nanotubes, and the lanthanoid is preferably gadolinium, europium, terbium, or a mixture of at least two of gadolinium, europium, and terbium. Preferably, the carbon-rich gas comprises 20 percent to 100 percent ethylene and 0 percent to 80 percent of an inert gas selected from the group consisting of nitrogen, helium, neon, and argon.

The carbon nanotubes provided by the method of the invention can comprise endohedral carbon nanotube complexes. Preferably, the carbon nanotubes of the endohedral carbon nanotube complexes are single-wall carbon nanotubes. The endohedral carbon nanotube complexes of the invention preferably comprise carbon nanotubes and encapsulated lanthanoid metal atoms. More preferably, the endohedral carbon nanotube complexes comprise an average of about 20 encapsulated lanthanoid atoms per endohedral carbon nanotube complex. Preferably, the lanthanoid is gadolinium, europium, and/or terbium. The endohedral carbon nanotube complexes are useful imaging contrast agents.

Thus, the invention provides an imaging contrast agent, comprising single-wall carbon nanotubes. The single-wall carbon nanotubes preferably comprise encapsulated metal atoms and/or ions. More preferably, the metal atoms and/or ions comprise at least one lanthanoid, and, most preferably, the lanthanoid is selected from the group consisting of gadolinium, europium, terbium, and mixtures of at least two of gadolinium, europium, and terbium. The single-wall carbon nanotubes preferably have an average diameter of 2 nm and an average length of 1 μm.

The invention also provides a method for imaging living tissue, comprising introducing an imaging contrast agent, comprising single-wall carbon nanotubes, into living tissue, placing the living tissue into a medical imaging device, and obtaining an image of the tissue. The medical imaging of the invention includes, but is not limited to, X-ray, computed tomography, single photon-emission-computed tomography, positron emission tomography (PET), magnetic resonance imaging (MRI), ultrasound imaging, radio frequency (rf), optical imaging, thermo-acoustic (TA) tomography (TAT), photo-acoustic (PA) tomography (PAT), and a combination of thermo-acoustic tomography, photo-acoustic tomography. Preferably, the medical imaging is a combination of thermo-acoustic tomography and photo-acoustic tomography. As discussed above, the single-wall carbon nanotubes preferably comprise encapsulated metal atoms and/or ions, where the metal atoms and/or ions more preferably comprise at least one lanthanoid, and, most preferably, the at least one lanthanoid is gadolinium, europium, terbium, or a mixture of at least two of gadolinium, europium, and terbium. Preferably, the single-wall carbon nanotubes used in the method of the invention have an average diameter of 2 nm and an average length of 1 μm.

### BRIEF DESCRIPTION OF THE FIGURES

FIG. 1a illustrates a Raman spectrum for Eu-catalyzed SWNTs (“Eu-SWNTs”);

FIG. 1b illustrates a Raman spectrum for Gd-catalyzed SWNTs (“G-SWNTs”);

FIG. 1c illustrates a Raman spectrum for the radial breathing modes for the Gd-SWNTs, ranging from 107 to 283 cm⁻¹, and corresponding to diameters between 0.8 and 2.3 nm;

FIG. 1d illustrates a Raman spectrum for the radial breathing modes for Eu-SWNTs, ranging from 163 to 289 cm⁻¹, and corresponding to diameters between 0.8 and 1.5 nm.

FIG. 2 illustrates TA and PA signals for controls.

FIG. 3 illustrates a comparison of TA and PA signals for SWNTs and controls.

FIG. 4 illustrates the emission spectra of Eu³⁺ ion from Gd—Eu-SWNTs.

### DETAILED DESCRIPTION OF THE INVENTION

The present invention is directed to a process for the preparation of carbon nanotubes, particularly single-walled carbon nanotubes, i.e., SWNTs, and to catalysts for the preparation of carbon nanotubes from a carbon-containing gas mixture. The catalyst of the invention comprises nanoclusters of at least one lanthanoid metal, previously known as a lanthanide metal, i.e., elements 57 to 71. Preferably, the catalyst comprises nanoclusters of at least one of the lanthanoids gadolinium, europium, and terbium. Mixtures of lanthanoid metals, preferably, mixtures of at least two of gadolinium, europium, and terbium, may also be employed as catalysts in the invention. The lanthanoid metals and mixtures of lanthanoid metals may also be used in combination with other metals known to catalyze SWNT formation and growth. Preferably, the catalysts are prepared by a self-organizing block copolymer templating technique. The catalytic nanoclusters of at least one lanthanoid metal typically have a diameter of between 1 to 5 nm, and are preferably 2 nm in diameter. It is understood that the nanoclusters may vary in dimension according to the method by which they are produced. Further the small values set forth are not absolute, but reflect measurements that have some degree of imprecision, or are values derived from such measurements.

The catalyst nanoclusters of the lanthanoid metal or metals are preferably disposed on the surface of a refractory support material. The support can be any solid material sufficiently refractory to withstand an oxygen plasma and CVD conditions, having wetting properties that induce phase separation of a block copolymer. Suitable support materials include, but are not limited to, mica, quartz, and silicon. Preferably, the support is silicon, and, more preferably, is a silicon having a thermal oxide layer supporting the nanoclusters.

The invention also provides a method for the formation of carbon nanotubes, preferably, SWNTs, the method comprising the step of contacting a carbon-rich gas with a catalyst of the invention at an elevated temperature. Suitable temperatures typically range from 600° to 900° C. The range is should not be considered absolute, but descriptive of useful temperature conditions. Useful temperatures will also be near to, but outside these boundaries. Further, the optimum temperature will vary with the metal or metals used in the catalyst and the nature of the carbon-rich gas. The determination of the optimum temperature will be readily determined by one of ordinary skill in the art.

The carbon-rich gas preferably comprises at least one hydrocarbon that can be provided as a gas or vapor at the temperature of the process of the invention. Suitable hydrocarbons include alkanes, such as methane and ethane, alkenes or olefins, such as ethylene, propylene, allene, and butadiene, and alkynes, such as acetylene and propyne, i.e., methylacetylene. A preferred hydrocarbon for use as the carbon-rich gas in the process of the invention is ethylene. More preferably, the carbon-rich gas comprises 20 to 100 mole percent ethylene and 0 to 80 mole percent of an inert gas, preferably, selected from the group consisting of nitrogen, helium, neon, and argon. As will be recognized by those skilled in the art, molar percentages are equivalent to volume percentages at standard temperature and pressure. In a preferred embodiment, the gas comprises 85 to 95 percent ethylene with the remainder an inert gas, where the inert gas is most preferably argon. In another preferred embodiment, the gas comprises about 90 percent ethylene and about 10 percent inert gas, where the inert gas is most preferably argon.

The lanthanoid metal catalyst nanoclusters are preferably prepared by phase separation of a block copolymer in which lanthanoid metal atoms are selectively bound to ligands in one of the polymer phases. A self-assembling block copolymer typically contains two or more different polymeric block components that are immiscible with one another. Under suitable conditions, the immiscible polymeric block components separate into two or more different phases on a nanometer scale, and form ordered patterns of isolated nano-sized structural units.

Such ordered patterns of isolated nano-sized structural units, formed by the self-assembling block copolymers, are used in the present invention for fabricating isolated nanoclusters of lanthanoid metals, for example, by selectively binding lanthanoid metal atoms in solution to suitable ligands of a component of a polymer block. Preferably, the ligand is pyridine, and the polymer block is poly-2-vinylpyridine. Spin-casting of the metal-containing block copolymer solution onto a suitable solid support is accompanied by phase separation into nanoscale domains, with the lanthanoid metal atoms selectively bound to the ligand-containing domains. The size of the domains, and thus the size of the metal nanoclusters, can be modified by altering the lengths of the copolymer blocks. Exposure to an oxygen plasma removes the polymer, leaving catalytic lanthanoid metal nanoclusters on the support surface. The chemical vapor deposition synthesis of carbon nanotubes is performed by contacting the catalytic lanthanoid metal nanoclusters with a carbon-rich gas at a temperature conducive to SWNT growth. The gas preferably contains ethylene, and the gas is contacted with the catalytic nanoparticles at a temperature of from about 700° to about 900° C., and, more preferably, about 750° C.

Nanoparticles of Gd and Eu, used in the growth of the SWNTs, were analyzed by atomic force microscopy (AFM). The AFM results demonstrate that the block copolymer templating method is suitable for obtaining large-area, uniform deposition of Gd nanoparticles with an average diameter of 1.9 nm and a narrow size distribution. Europium, i.e., Eu nanoparticles, prepared using the same block copolymer templating method, provided similar results.

An analysis of SWNT bundles grown on the Gd and Eu nanoparticles by low resolution transmission electron microscope (TEM) indicated Gd and Eu catalyzed SWNTs having diameters of 2.05 nm and 2.10 nm respectively.

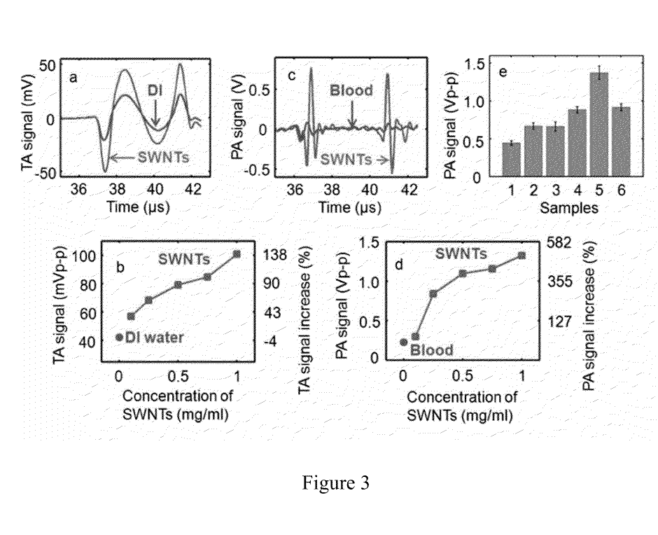

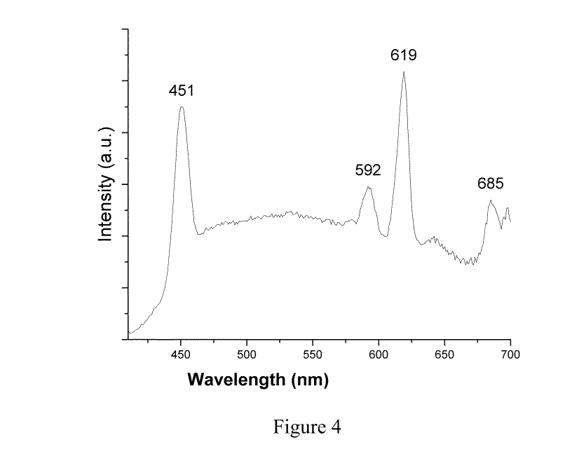

In addition to TEM and AFM, the growth of SWNTs was further confirmed by Raman spectroscopy, as illustrated in FIG. 3. Raman spectroscopy is an important characterization tool for carbon nanotubes, providing information on the diameter, purity, and electronic properties of SWNTs.

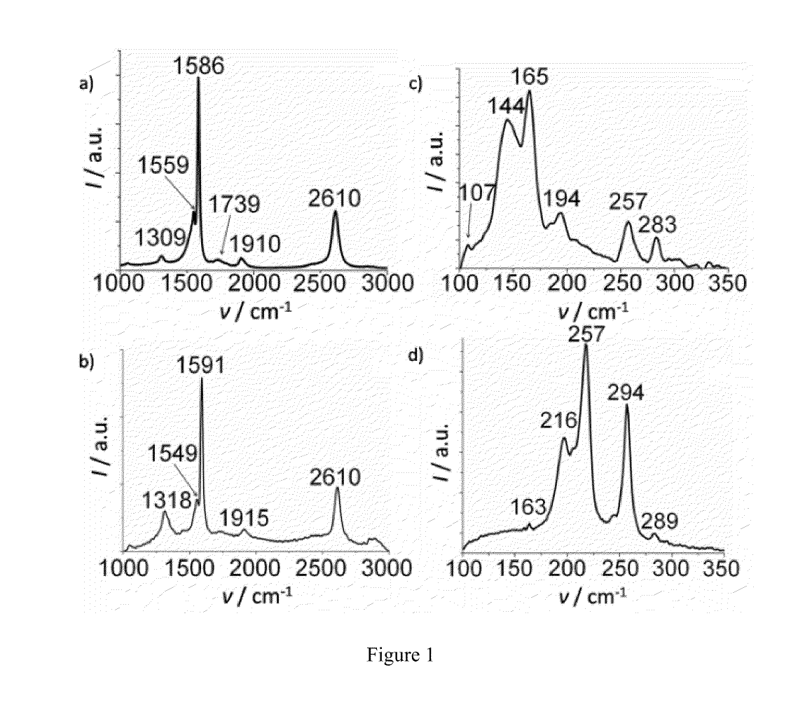

The Raman spectrum for the Gd-catalyzed SWNTs (hereinafter referred as Gd-SWNTs), illustrated in FIG. 1b, has a G band at 1591 cm⁻¹ with a downshifted G− band at 1549 cm⁻¹. The G band is a single Lorentzian peak for graphite due to the tangential mode vibrations between C atoms. When curvature exists, as in a SWNT, vibrations in the circumferential direction form two components of the G band; i.e., G+ at 1590 cm⁻¹ and G− at 1570 cm⁻¹. The G band wavenumber, however, is variable, and decreases with decreasing SWNT diameters. Therefore, the downshifted G− band illustrated in FIG. 1b for Gd-SWNTs corresponds to relatively small diameter SWNTs.

The G− band for Gd-SWNTs has a Lorentzian line shape, as illustrated in FIG. 1a, and, thus, is characteristic of a semiconducting SWNT. The additional Raman peaks, such as D, iTOLA, and G′ present in the Raman spectrum illustrated in FIG. 1b are due to double resonance, and result from elastic scattering caused by defects in the nanotubes or inelastic scattering by phonon emission. The D band of the Raman spectrum illustrated in FIG. 1b at 1318 cm⁻¹ is a disorder-induced mode in graphite, and usually occurs when there are defects in the SWNTs. Therefore, the D band is considered a measure of sample purity. The D/G band ratio in the illustrated spectrum for the Gd-SWNTs is about 0.11, demonstrating that only a small proportion of the SWNTs have any defects.

The G′ band at 2610 cm⁻¹ in the spectrum illustrated in FIG. 1b has a wavenumber approximately twice that of the wavenumber of the D band. The G′ band at 2610 cm⁻¹ is known to be an overtone mode of the D band. Unlike the D band, the G′ mode is not known to occur as a result of SWNT defects. The iTOLA band at 1915 cm⁻¹ results from a double resonance not found in graphite, and is due to the combination of the in-plane transverse optical branch (iTO) and the second phonon from the longitudinal acoustic branch (LA), and, therefore, is called a combination phonon mode. Although this band exhibits high experimental dispersion, the occurrence of the double resonance process further corroborates the existence of SWNTs, as set forth in Dresselhaus et al., Raman Spectroscopy of Carbon Nanotubes, Physics Reports 2005; 409(2):47-99, and Thomsen et al., Raman Scattering in Carbon Nanotubes, Light Scattering in Solid IX 2007:115-234.

FIG. 1a illustrates the Raman spectrum of Eu-catalyzed SWNTs (hereinafter referred as Eu-SWNTs). The Raman spectrum of the Eu-SWNTs has a G band at 1586 cm⁻¹ with a G− band at 1559 cm⁻¹, where each band has a Lorentzian lineshape. The Raman spectrum of the Eu-SWNTs has an additional peak at 1739 cm⁻¹ corresponding to the M band, which is an overtone of a graphite mode, emphasized by the curvature of the nanotubes. The double resonance D, iTOLA, and G′ bands for the Eu-SWNTs are slightly down-shifted compared to corresponding bands for the Gd-SWNTs, and occur at 1309 cm⁻¹, 1910 cm⁻¹, and 2610 cm⁻¹, respectively. The D/G ratio for the Raman spectrum of the Eu-catalyzed SWNTs is 0.04, again indicating nearly defect-free SWNTs.

The radial breathing modes (RBM), found in the 100 to 350 cm⁻¹ region of the Raman spectra, are unique to SWNTs, and occur at frequencies specific to nanotube diameters. Since the SWNTs form bundles, van der Waals interactions among the tubes affect the linear relationship between the radial breathing mode frequencies and the diameter of the nanotubes. Thus, using the relationship dₜ=[238 cm⁻¹ nm/ω_{RBM}]^{1/0.93}, where dₜ is the diameter of the tube in nanometers, and ω_{RBM} is the wavenumber of the Raman spectral peak that corresponds to dₜ, it is possible to calculate a value of dₜ for the SWNTs.

The radial breathing modes values for the Gd-SWNTs obtained from the spectrum illustrated in FIG. 1c range from 107 to 283 cm⁻¹. Those values correspond to nanotube diameters between about 0.8 and about 2.3 nm. Similarly, the radial breathing modes values for the Eu-SWNTs obtained from the spectrum illustrated in FIG. 1d range from 163 to 289 cm⁻¹. Those values correspond to nanotube diameters to diameters between about 0.8 and about 1.5 nm.

Kataura plots are graphs relating the energy of the band gaps in a carbon nanotube and the diameter of nanotube, and are routinely used to determine whether a SWNT is metallic or semiconducting, as a function of the nanotube diameters. van-Hove singularities in the joint density of states with a third order tight-binding approximation were used to obtain the desired information. Kataura plots were used to determine the properties of the Gd-SWNTs and Eu-SWNTs described above using the diameters obtained from the radial breathing modes. The obtained plots demonstrate that, at the laser excitation energy of 1.96 eV, the Gd-SWNTs, having prominent radial breathing modes at 107, 144, 165, and 283 cm⁻¹, contain semi-conducting SWNTs as the major fraction, and the Eu-SWNTs, having prominent radial breathing modes at 216, 257, and 294 cm⁻¹, contain metallic SWNTs respectively as the major fraction. It is believed that additional spectra collected at different laser wavelengths will identify and confirm the electronic properties of the SWNTs based on the RBM.

### Examples

### Catalyst Preparation

Catalysts were prepared from gadolinium (III) chloride, europium (II) chloride, and terbium(III) chloride. The catalysts were deposited using the method disclosed by Fu et al. (Q. Fu, S. Huang and J. Liu, J. Phys. Chem. B, 2004, 108:6124-6129), as follows: A 1 mg/ml solution of polystyrene-poly(2-vinylpyridine) block copolymer (PS Mₙ 53400, P2VP Mₙ 8800) (“PS-b-P2VP”) (product P118-S2VP; Polymer Source, Montreal, Canada) in toluene (Fisher Scientific) was prepared by stirring the polymer in toluene for 5 hours. Gadolinium (III) chloride, europium (II) chloride, terbium (III) chloride, or a mixture of two of those compounds (4 mg) was then added to 5 ml of the polymer solution with a molar loading ratio of metal to 2-vinylpyridine of 1:1, and stirring for an additional 24 hours. The solution was then spin-coated at 6100 rpm for 1 minute onto a p-type doped Si wafer with a 1000 nm thermal oxide layer (University Wafers, Boston, Mass.). The coated wafer was then exposed to an oxygen plasma in an oxygen plasma cleaner (model PDC-32G, Harrick Plasma, Ithaca, N.Y.) with 700V applied to the RF coil, at about 1 mbar for 15 minutes.

### Chemical Vapor Deposition

Carbon nanotubes were synthesized by chemical vapor deposition in a CVD furnace (Easy Tube™ 2000, First Nano, Ronkonkoma, N.Y.) following procedures as generally described in U.S. Patent Application Publication No. 2008/0095695, incorporated herein by reference in its entirety. The catalyst substrate described above was placed in a 3 inch quartz reaction chamber, heated in argon to 750° C., and soaked in hydrogen for 2 minutes. The SWNT synthesis was initiated by adding ethylene to the gas flow for 20 minutes. After growth, the carbon feedstock was switched off, and the furnace cooled to room temperature.

### Raman Spectroscopy

The Raman spectral analysis was performed with a LabRAM HR (Horiba JobinYvon, Edison, N.J.) at 633 nm excitation. Transmission electron microscopy (TEM) was performed on the samples with a Tecnail2 BioTwinG2 (FEI, Hillsboro, Oreg.) at 80 kV. Digital images were acquired with an AMT XR-60 CCD Digital Camera System. Tapping mode Atomic Force Microscopy (AFM) measurements of the catalysts and carbon nanotubes were obtained with an Asylum MFD-3D-BIO (Asylum Research, Santa Barbara, Calif.).

As illustrated in FIGS. 1c and 1d, the radial breathing modes for the Gd-SWNTs and Eu-SWNTs differ. The radial breathing modes for the Gd-SWNTs, illustrated in FIG. 1c, range from 107 to 283 cm⁻¹, and correspond to diameters between 0.8 and 2.3 nm. The radial breathing modes for the Eu-SWNTs, illustrated in FIG. 1d, range from 163 to 289 cm⁻¹, and correspond to diameters between 0.8 and 1.5 nm.

Metal analysis by ICP

The amount of lanthanoid, such as Gd, Eu, or Tb, encapsulated in the single-walled carbon nanotube formed, as described above, was determined using inductively-coupled plasma spectroscopy (ICP), the method of choice for quantifying trace metal content. In preparation for the ICP measurements, Gd-SWNT and Eu-SWNT solutions were treated with ca. 90 percent HNO₃, then carefully heated until a solid residue was obtained. The mixtures were then further treated with a 30 percent H₂O₂ solution, and again carefully heated to completely remove any remaining carbonaceous material. The solid residue was dissolved in 2 percent HNO₃ and analyzed by ICP.

ICP analysis was performed on a Varian Vista Pro Simultaneous Axial Inductively Coupled Atomic Emission Spectrometer (ICP-OES) with a CCD detector. Gd lines at 335.05 nm, 342.35 nm and 376.84 nm were analyzed. Seven scans were performed for each sample, providing data having a relative standard deviation (RSD) of 0.2 percent. The Gd line at 376.84 has a higher intensity, and was chosen for the determination of the Gd concentration. Sc (λ=361.38 nm) was used as the internal drift standard. The results are provided in Table 1.

TABLE 1
Endohedral Metals Quantitation

| | Metal content (Gd or Eu) |
|---|---|
| Sample | μg/ml (parts per million) |
| Gd-SWNT grown on 3 inch Si Wafers | 10.0 ± 1.1 |
| Gd-SWNT grown on 6 inch Si Wafers | 22.1 ± 3.8 |
| Gd-SWNT grown on 10 inch Si Wafers | 35.2 ± 2.5 |
| Eu-SWNT grown on 3 inch Si Wafers | 9.2 ± 1.1 |
| Eu-SWNT grown on 6 inch Si Wafers | 24.6 ± 3.8 |
| Eu-SWNT grown on 10 inch Si Wafers | 39.5 ± 2.5 |

Using equations by K. Yamamoto et al., Japan. J. Appl. Phys. Part 1-Regular Papers Short Notes & Review Papers, 2005, 44:1611-1614, the number of carbon atoms in a carbon nanotube having an average diameter of 2 nm and an average length of 1 μm is about 10⁵ carbon atoms, corresponding to an average molecular weight of ca. 10⁶ Da. The atomic radius of Gd is 0.107 nm, the atomic radius of Eu is 0.108 nm, and the average size of a catalytic Gd or Eu nanocluster is 1.9 nm with a standard deviation of less than 10 percent. Those figures demonstrate that each SWNT grows on one catalyst nanocluster, and encapsulates on average ca. 20 Gd and/or Eu atoms.

The successful demonstration of SWNT growth from Gd, Eu, and Tb, and the ready formation of endohedral complexes with these metals, opens avenues for their physiochemical characterization. In particular, Gd, Eu, and Tb possess magnetic and photoluminescent properties that can be exploited, as a result of the unique characteristics of an enclosing SWNT, resulting in the development of a wide range of biomedical applications.

For example, Eu-SWNTs exhibit bright red fluorescence in the visible region at an emission wavelength of 619 nm, following excitation at a wavelength of 380 to 415 nm, and Tb-SWNTs exhibit bright green fluorescence in the visible region at an emission wavelength of 550 nm, following excitation at a wavelength of 380 to 415 nm. FIG. 3 illustrates the emission spectra of Eu³⁺ ion from Gd—Eu-SWNTs. The result demonstrates that Eu³⁺ and Tb³⁺ have energy acceptor levels that are suitable for energy transfer in the visible regions, e.g., for excitation wavelengths of 380 to 450 nm, and overlap with the broad absorption band of SWNTs, which act as energy donors that transfer the absorbed energy from the SWNT to the Eu³⁺ ion. Here, SWNTs serves as antennae or sensitizers to absorb excitation light and transfer this energy to lanthanoid Eu ions that have inherently weak absorbance, i.e., 1 M⁻¹ cm⁻¹, or a factor of 100 smaller than conventional organic dyes. SWNTs also tightly bind the lanthanoid, sequestering lanthanoid ion toxicity, shielding the lanthanoid ion from the quenching effects of water, and acting as a scaffold for attachment to biomolecules. Spectral characteristics of Eu-SWNTs and Tb-SWNTs are similar to those of lanthanoid chelates, having long a fluorescence lifetime in the sub-microsecond to millisecond range, sharply spiked emission spectra of less than 10 nm full width at half-maximum, large Stokes shifts of greater than 150 nm, and a high quantum yield of about 1. Those properties make SWNTs useful alternatives to organic dyes, particularly where problems of background auto-fluorescence exist, as donors in luminescence resonance energy transfer experiments, and as structural/functional probes in biological systems.

With the invention, magnetic materials, such as Gd, can be encapsulated within SWNTs, e.g., Gd—Eu-SWNTs. T₁-weighted MRI phantom studies of paramagnetic Gd—Eu-SWNTs at a Gd⁺³ concentration of 100 nM demonstrated that Gd—Eu-SWNTs have extremely high signal enhancement compared to the clinically used Gd-chelated contrast agent Magnevist™ at the same concentration, with relaxivities 100 fold greater than Magnevist™, and 3 to 5 fold greater than other carbon nanostructure based contrast agents, as demonstrated in see Table 2. Table 3 provides a comparison of the T₁ relaxivities of Gd—Eu-SWNTs to clinically used Gd-chelated contrast agent Magnevist™ and other carbon nanostructure-based contrast agents, such as gadofullerenes, i.e., Gd-C60 Gd-C80 and Gd-C82, and Gd-nanotubes, i.e., Gd-US-tubes, that encapsulate Gd atoms (T=25° C., magnetic field=3 Tesla). The r₂ relaxivity of the Gd—Eu-SWNTs is 871 mM⁻¹ s⁻¹ at 3 Tesla. The results demonstrate that Gd—Eu-SWNTs are suitable as T₁ contrast agents, and have the ability to generate high contrast at low concentrations where clinical Magnevist™ has little, if any, noticeable enhancement, and, thus, are strong candidates for advanced in vivo molecular MRI.

TABLE 2

| Contrast Agent | r₁ (mM⁻¹ · s⁻¹) |
|---|---|
| Gd-Eu-SWNT | 459 |
| Gadofullerenes | 10 to 95 |
| Gadonanotubes | 130 to 170 |
| Magnevist ™ | 4.5 |

SWNTs have been found to be useful as carbon nanotube-based contrast agents for a variety of imaging techniques. For use as carbon nanotube-based contrast agents, the SWNTs encapsulate medically relevant metal ions, such as those of Gd and Ep, within the carbon sheath. The intrinsic optical and rf absorbing properties of SWNTs have been found to be useful as multimodal contrast agents for simultaneous thermo-acoustic (TA) tomography (TAT) and photo-acoustic (PA) tomography (PAT).

A combined TAT/PAT scanner was used to test the SWNTs as carbon nanotube-based contrast agents for TAT/PAT. Images were collected with microwave excitation (TAT), and then with laser excitation (PAT). For TAT, a 3.0-GHz microwave source with a 0.5 μs pulse width and 100-Hz pulse repetition rate was the rf source. The pulse energy was estimated to be around 10 mJ, i.e., 20 kW×0.5 μs, falling within the IEEE safety standards. PAT measurements were performed at a wavelength of 1064 nm, produced by a 1064 nm wavelength Q-switched Nd:YAG laser with a 10-Hz pulse repetition rate, 6.5 ns laser pulse width, and 850 mJ maximum output energy. The incident laser fluence on the sample surface was controlled to be less than 20 mJ/cm², conforming to the American National Standards Institute (ANSI) standards.

The generated acoustic signals were detected using two different non-focused transducers, operating at a central frequency of 2.25 MHz (13-mm-diam active area, ISS 2.25×0.5 COM; 6-mm-diameter active area, ISS 2.25×0.25 COM, Krautkramer). For cross-sectional TAT/PAT imaging, data were collected around the sample in a full circle. A delay and sum (back projection) algorithm was used for all image reconstruction. The samples were placed inside a breast holder chamber, filled with mineral oil, as mineral oil does not absorb microwaves. Moreover, because mineral oil is transparent, the light absorption is also negligible. Mineral oil also acts as a coupling medium for sound propagation. Thus, mineral oil is well suited as a background medium.

As well as SWNTs, the imaging properties of four types of multi-walled carbon nanotubes (MWNTs) of various inner and outer diameters, fullerenes (Sigma-Aldrich, USA, catalog number 483036), and graphite micro-particles (Sigma-Aldrich, catalog number 496596) were determined. SWNT suspensions with concentrations ranging from 0.1 to 1 mg/ml were prepared in 10 ml of 1 percent biologically compatible Pluronic® F127 surfactant solution, having a pH of 7. Without being bound by theory, it is believed that the different domains of the nonionic Pluronic® F127 wrapped themselves around the nanotubes in energy-minimized conformations to solubilize the SWNTs by steric stabilization, producing nearly neutral nanotube suspensions. The suspensions were found to be stable over the time period required for the measurements.

ζ-potential measurements were performed on dispersions of SWNTs dispersed in Pluronic® F-127 at a concentration of 0.1 mg/ml, and had a peak ζ-potential of −14 mV with a Gaussian distribution, i.e., the full width half maximum of the distribution was 10 mV. That value is similar to other reported ζ-potential measurements on neutral stable SWNTs dispersed in Pluronic® F127.

A reflection-mode photo-acoustic imaging system was used to determine the in vitro blood signal enhancement using SWNTs. A tunable Ti:sapphire laser (LT-2211A, LOTIS TII) pumped by a Q-switched Nd:YAG (LS-2137, LOTIS II) laser was used as the light source, providing a pulse duration of less than 15 ns and a 10-Hz pulse repetition rate. A 5 MHz central frequency, spherically focused ultrasonic transducer (V308, Panametrics-NDT) was used to acquire the generated PA signals. The transducer had a 2.54 cm focus length, a 1.91 cm diameter active area element, and a 72 percent bandwidth. The signal was then amplified by a low-noise amplifier (5072PR, Panametrics-NDT), and recorded using a digital oscilloscope (TDS 5054, Tektronix) with a 50 mega-sampling rate. PA signal fluctuations due to pulse-to-pulse energy variation were compensated by signals from a photodiode (DET110, Thorlabs), which sampled the energy of each laser pulse.

A low-density polyethylene (LDPE) vial, having an inner diameter of 6 mm and a 1 cm³ volume was filled with the sample, and placed inside the TAT/PAT scanner. Deionized (DI) water was used for TA signal comparison, and blood was used for PA signal comparison. Water and ions are two well-known sources of microwave absorbers in the human body, and produce strong TA signals. It was demonstrated that SWNTs can function as a contrast agent by generating TA signals comparable to or stronger than a known TA signal producer in the body. The rf contrast between malignant tumor tissue and normal human breast tissue is as high as 4:1. The rf absorption of water compared to background human breast tissue is also on the order of 4:1. By comparing the rf absorption of SWNTs to that of water, the ability of SWNTs to function as contrast agents for TA was confirmed.

Similarly, blood is a dominant light absorber in the human body, and produces strong PA signals. Therefore, it was demonstrated that SWNTs can function as a contrast agent in PA by generating PA signals comparable to or stronger than that of a known absorber in the body, such as blood. For the tissue phantom imaging, porcine fat was used as the background medium mimicking the tissue. The sample holder, i.e., the LDPE vial, was inserted in the center of a porcine fat cylinder having a diameter of about 6-cm diameter and a height of about 1.5 cm. The vial was filled with samples of deionized water, SWNTs, and blood, and images were collected. The hole inside the porcine fat had a slightly larger diameter than the sample holder's outside diameter.

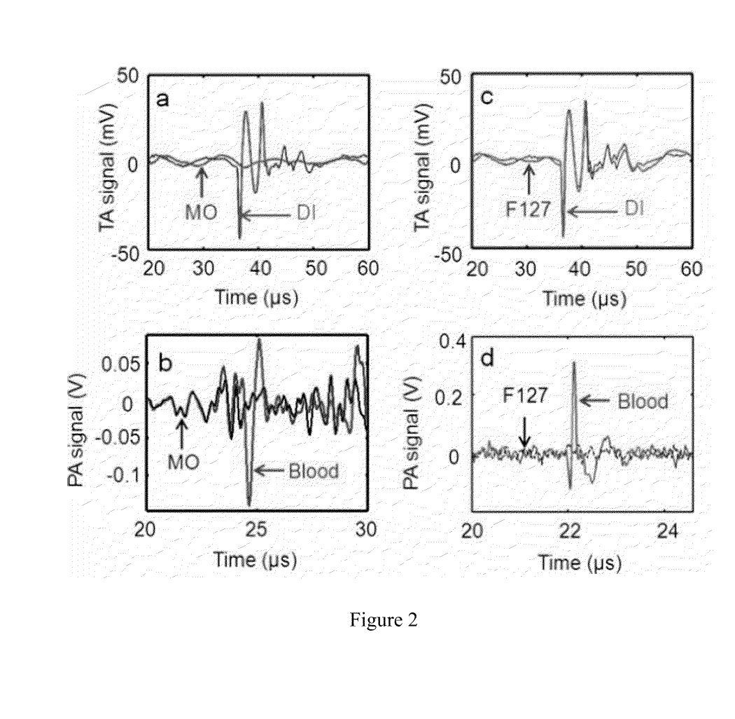

FIG. 2(a) illustrates the TA signal generated from the sample holder (LDPE vial) filled with deionized water and with mineral oil. No TA signal was observed, demonstrating that the LDPE vial does not generate a TA signal. As the LDPE vial used was semi-transparent white, it did not absorb enough light to produce a measurable PA signal. The sample holder was further tested by filling the LDPE vial with blood and mineral oil under 1064-nm wavelength light. FIG. 2(b) illustrates the PA signal generated from the vial. No significant PA signal was observed at 1064 nm compared to the signal generated from blood, further demonstrating that the sample holder vial had no effect on TA or PA signal generation.

A 1 percent Pluronic® F127 surfactant solution was also tested, and no significant TA/PA signals were observed. FIG. 2(c) illustrates the TA signal generated from the LDPE vial filled with 1 percent Pluronic® F127 surfactant solution and deionized water, and no significant contribution from the surfactant solution was observed. FIG. 2(d) illustrates the PA signal generated from blood and 1 percent Pluronic® F127 surfactant solution. No PA signal was generated from 1 percent Pluronic® F127 surfactant solution.

An initial assessment was made for SWNTs, MWNTs, C₆₀, and graphite microparticles. Table 3 summarizes the peak-to-peak TA/PA signal amplitudes obtained from various samples with the two different diameter transducers.

TABLE 3

| | Peak-to-peak TA signal | | Peak-to-peak PA signal | |
|---|---|---|---|---|
| | amplitude (mV) | | amplitude (mV) | |
| | 0.5-in. active | 0.25-in. active | 0.5-in. active | 0.25-in. active |
| | area | area | area | area |
| Sample | transducer | transducer | transducer | transducer |
| Deionized water | 47 | 14 | — | — |
| SWNT | 95 | 28 | 113 | 81 |
| (1 mg/mL) | | | | |
| Fullerene (C₆₀) | 51 | 15 | 55 | 68 |
| Graphite microparticles | 38 | 10 | 28 | 30 |
| MWNT | 40 | 12 | 95 | 81 |
| (o.d. = 10-15 nm) | | | | |
| Aldrich MWNT | 40 | 12 | 58 | 67 |
| (o.d. = 20-30 nm) | | | | |
| MWNT | 39 | 10 | 28 | 27 |
| (o.d = 40-70 nm) | | | | |
| MWNT | 47 | 15 | 62 | 70 |
| (o.d. = 110-170 nm) | | | | |
| Blood | — | — | 33 | 31 |

Only SWNTs demonstrated a significant increase in TA signal compared to deionized water and a significant increase in PA signal compared to rat blood. It should be noted that TA and PA signals generated from MWNTs are not directly proportional to the outer diameter. Other parameters, such as the inner diameter, nanotube length, and number of concentric nanotubes, can also affect the generated signal amplitudes. However, the data demonstrates that SWNTs generate a PA and TA signal stronger than that of blood, water, and other carbon nanostructures.

FIG. 3(a) illustrates the TA signals from an LDPE vial filled with deionized water and a second vial filled with 1 mg/ml SWNTs. The peak-to-peak TA signal amplitudes generated by deionized water and 1 mg/ml SWNTs are 42±0.32 and 101±0.24 mV, respectively.

FIG. 3(b) illustrates the peak-to-peak TA signal amplitude and fractional increase in TA signal versus the concentration of SWNTs. The largest standard deviation of the data points, measuring 0.92 mV, was observed for a 0.75 mg/ml SWNT concentration. The data demonstrate an approximately linear relationship between the TA signal amplitude and the SWNT concentration. A maximum of 140 percent increase in the peak-to-peak signal amplitude was observed for 1 mg/ml SWNTs over deionized water.

FIG. 3(c) illustrates the PA signals from a LDPE vial filled with blood and with 1 mg/ml SWNTs. The peak-to-peak PA signal amplitudes generated by blood and the 1 mg/ml SWNTs are 0.22±0.002 and 1.32±0.009 V, respectively.

FIG. 3(d) illustrates the peak-to-peak PA signal amplitude and fractional increase in PA signal versus the concentration of SWNTs. The largest standard deviation of the data points, measuring 0.027 V, was again observed at a 0.75 mg/ml SWNT concentration.

The data again demonstrate an approximately linear relationship between the PA signal amplitude and the SWNT concentration. A maximum 490 percent increase in the peak-to-peak signal was for 1 mg/ml SWNTs compared to that of blood.

In vitro tests were carried out with a SWNT concentration of 0.1 mg/ml mixed with blood in different proportions, and the PA signals were recorded. Near infra-red light (NIR), i.e., 700 to 800 nm, is typically used for in vivo deep tissue imaging. In the present case, the light used in the reflection mode PA imaging system had a wavelength of 754 nm. A tube (Silastic® laboratory tubing, Dow Corning Corp., having a 300 μm i.d. and a 640 μm o.d.) was filled with blood, blood (90 percent v/v)+SWNTs (10 percent v/v), blood (75 percent v/v)+SWNTs (25 percent v/v), blood (50 percent v/v)+SWNTs (50 percent v/v), blood (25 percent v/v)+SWNTs (75 percent v/v), and SWNTs alone.

FIG. 3(e) illustrates the peak-to-peak PA signal amplitudes for those six samples, demonstrating that the PA signal from blood was enhanced when SWNTs were mixed with the blood. The measurements were repeated 10 times, providing an average PA signal of 1.37±0.09 V from a mixture of 75 percent SWNTs and 25 percent blood, compared to a 0.44±0.02 V PA signal from only blood. Therefore, when SWNTs were mixed with the blood, at least a 210 percent increase in the PA signal was observed at a wavelength of 754 nm.

LDPE vials filled with different SWNTs samples were imaged in two dimensions using TAT and PAT. The TAT cross-sectional image of vials filled with deionized water and SWNTs, respectively, demonstrated a substantial improvement in the TA signal for the vial filled with SWNTs. Compared to deionized water, SWNTs had about 1.9 to about 2.1 times signal improvements in the TA image (normalized to deionized water). These results are consistent with the TA data presented in FIG. 3. Cross-sectional PAT images of vials filled with blood and SWNTs, respectively, demonstrate an increased signal in the PAT images with SWNTs compared to blood. Compared to blood, SWNTs have about a 5.6 to about 6.3-fold signal improvement (normalized to blood). The results are consistent with the PA data presented in FIG. 3. The variation in the signal improvement along the horizontal and vertical directions for both TAT and PAT is possibly due to the anisotropic spatial resolution.

The images obtained demonstrated greater contrast for the SWNTs than for the deionized-water-filled vial. PAT images for blood and SWNTs, respectively, for the tissue phantom provide results similar to those obtained by TAT imaging. That is, the SWNT sample provided greater contrast than blood.

SWNTs have been shown to generate both TA and PA signals, and SWNTs can be used for TA/PA imaging, as the intrinsic absorption properties of SWNTs are able to produce equivalent or stronger TA/PA signals, depending on the SWNTs concentration, than known endogenous absorbers, i.e., more than water in the case of TAT, and more than blood in the case of PAT. In addition, a substantial enhancement in the PA signal was obtained in vitro when SWNTs were mixed with blood. Thus, when a SWNT solution is mixed with water/blood the SWNTs, the SWNTs enhance the total TA/PA signal from the water/blood.

In addition, TA/PA signals generated by SWNTs can be used for imaging applications where no endogenous signals are found. For example, PA imaging and SWNTs allow in vivo noninvasive sentinel lymph-node mapping. Although no blood signal present, the PA signals generated by the SWNTs allowed imaging sentinel lymph nodes. Another example is a targeted molecular imaging application, where the TA/PA signal is generated from the contrast agents themselves.

The optical absorption properties of SWNTs are strong in the visible and NIR region. Currently, no studies have demonstrated SWNTs' absorption property in the 3-GHz microwave region, but their conductive properties make them promising for strong absorption. SWNTs have high permittivity when exposed to electromagnetic radiation at frequencies between 0.5 and 3 GHz, and, as the frequency increases to greater than 3 GHz, the permittivity decreases. SWNTs can be used as contrast agents at less than 3 GHz.

Thus, SWNTs are suitable as a contrast agent for both TAT and PAT. The SWNTs allow contrast-enhanced deep-tissue imaging with TAT, providing contrast enhancement at an rf frequency of 3 GHz. As the human body becomes more transparent at lower rf frequencies, it is expected that lower rf frequencies will provide an increase in the imaging depth. However, because of lower tissue absorbance at lower rf frequencies, the intrinsic image contrast suffers. Therefore, as SWNTs work as a contrast agent at lower rf frequencies, SWNTs provide low background, high-sensitivity, deep-tissue imaging.

The broad absorption range of SWNTs in the visible/NIR region is also beneficial for optical imaging, as one can use a wide range of laser wavelengths for imaging without the need to tune the contrast agent to a particular wavelength to optimize light absorption. In comparison, other contrast agents suitable for PAT, such as gold nanoparticles, are tuned to a particular wavelength range, and can be used only with light within that range.

In addition, a minimum detectable concentration of SWNTs is expected to be comparable to that of gold nanoparticles. As stated above, carbon nanotubes of 2 nm average diameter and 1 μm average length have about 10⁵ carbon atoms, giving an average molecular weight of about 10⁶ Da or g/mol (multiply the number of carbon atoms by 12, the atomic weight of carbon). From FIG. 3, it is clear that the minimum detectable concentration is less than 0.1 mg/ml or 100 nM (0.1 mg/ml/10⁶ g/mol), 1 M SWNTs concentration, allowing their detection in the nM range.

It is also evident that, even at a 0.1 mg/ml SWNT concentration, there is a 35 percent increase in peak-to-peak TA signal compared to deionized water and a 32 percent increase in peak-to-peak PA signal compared to blood. For targeted molecular imaging applications, an important consideration is not only the enhancement, but also the signal generated by the SWNTs themselves. Signals with very high signal-to-noise ratio (SNR greater than 100 in both TA and PA) at 1 mg/ml SWNT concentration have been detected, providing a minimum detectable SWNT concentration as low as 0.01 mg/ml or about 10 nM, making SWNTs suitable for in vivo applications in various tissues. In general, the minimum detectable concentration of an exogenous contrast agent by PAT/TAT is dependent on many factors, such as incident light/microwave energy, ultrasound detector sensitivity, data acquisition electronics, etc. The concentration of SWNTs can depend on the specific application and the sensitivity of the imaging system.

SWNTs provide a number of additional benefits as contrast agents:

(i) Other than TAT and PAT, SWNTs can also be used as a contrast agent for other imaging modalities, such as MRI, PET, NIR optical imaging, and nuclear imaging, and, thus, work as a multi-modal contrast agents;

(ii) The external carbon sheath of the SWNTs can be directly functionalized for targeting and drug delivery, which is not possible for other optical contrast agents, such as gold nanoparticles, where one does not functionalize the gold, but rather the capping agents or the biocompatible coating used to stabilize and/or dispense gold nanoparticles in solution; and

(iii) SWNTs provide TAT and/or PAT molecular imaging with simultaneous therapy by NIR and rf-induced hyperthermia, as SWNTs have been shown to facilitate the NIR and rf-induced ablation of tumor cells/tissues, allowing the design of multimodal imaging and multitherapeutic approaches within a single platform.

Thus, SWNTs provide more than a two-fold signal enhancement in TAT at 3 GHz and more than six-fold signal enhancement in PAT at 1064 nm. That indicates that, by using SWNTs as contrast agents, the functional information from TAT and PAT together with other structural imaging modalities will be advantageous for early cancer diagnosis. At lower rfs, these exogenous contrast agents offer a new paradigm for low-background, high sensitivity, deep-tissue, and targeted molecular imaging by TAT.

What is claimed:

1. A catalyst for the formation of carbon nanotubes from a carbon-containing gas mixture, comprising nanoclusters of at least one lanthanoid metal.

2. The catalyst of claim 1, wherein the at least one lanthanoid is selected from the group consisting of gadolinium, europium, and terbium.

3. The catalyst of claim 1, wherein the nanoclusters are disposed on the surface of a refractory support material.

4. The catalyst of claim 3, wherein the support material is selected from the group consisting of mica, quartz, and silicon.

5. The catalyst of claim 4, wherein the support material is silicon.

6. A method for the formation of carbon nanotubes, comprising
providing a catalyst comprising nanoclusters of at least one lanthanoid metal;
contacting a carbon-rich gas with the catalyst; and
forming carbon nanotubes.

7. The method according to claim 6, wherein the carbon nanotubes are single-wall carbon nanotubes

8. The method according to claim 6, wherein the at least one lanthanoid is gadolinium, europium, terbium, or a mixture comprising at least two of gadolinium, europium, and terbium.

9. The method according to claim 6, wherein the nanoclusters are disposed on the surface of a refractory support material.

10. The method according to claim 8, wherein the support material is selected from the group consisting of mica, quartz, and silicon.

11. The method according to claim 8, wherein the support material is silicon.

12. The method of claim 6, wherein the carbon-rich gas comprises 20 to 100 mole percent ethylene and 0 to 80 mole percent of an inert gas selected from the group consisting of nitrogen, helium, neon, and argon.

13. The method according to claim 6, wherein the carbon nanotubes comprise endohedral carbon nanotube complexes.

14. The method according to claim 13, wherein the carbon nanotubes of the endohedral carbon nanotube complexes are single-wall carbon nanotubes.

15. Endohedral carbon nanotube complexes, comprising carbon nanotubes and encapsulated lanthanoid metal atoms.

16. The complexes according to claim 15, wherein the endohedral carbon nanotube complexes comprise an average of about 20 encapsulated lanthanoid atoms per endohedral carbon nanotube complex.

17. The complexes according to claim 15, wherein the lanthanoid is selected from the group consisting of gadolinium, europium, terbium, and mixtures comprising at least two of gadolinium, europium, and terbium.

18. The complexes according to claim 15, wherein the endohedral carbon nanotube complexes are imaging contrast agents.

19. An imaging contrast agent, comprising single-wall carbon nanotubes.

20. The imaging contrast agent according to claim 19, wherein the single-wall carbon nanotubes comprise encapsulated metal atoms and/or ions.

21. The imaging contrast agent according to claim 20, wherein the metal atoms and/or ions comprise at least one lanthanoid.

22. The imaging contrast agent according to claim 20, wherein the lanthanoid is selected from the group consisting of gadolinium, europium, terbium, and mixtures of at least two of gadolinium, europium, and terbium.

23. The imaging contrast agent according to claim 19, wherein the single-wall carbon nanotubes have an average diameter of 2 nm and an average length of 1 μm.

24. A method for imaging living tissue, the method comprising introducing an imaging contrast agent, comprising single-wall carbon nanotubes, into living tissue;
placing the living tissue into a medical imaging device; and
obtaining an image of the tissue.

25. The method according to claim 24, wherein the medical imaging device is selected from the group consisting of X-ray, computed tomography, single photon-emission-computed tomography, positron emission tomography (PET), magnetic resonance imaging (MRI), ultrasound imaging, radio frequency (rf), optical imaging, thermo-acoustic (TA) tomography (TAT), photo-acoustic (PA) tomography (PAT), and a combination of thermo-acoustic tomography and photo-acoustic tomography devices.

26. The method according to claim 24, wherein the medical imaging is device combines thermo-acoustic tomography and photo-acoustic tomography.

27. The method according to claim 24, wherein the single-wall carbon nanotubes comprise encapsulated metal atoms and/or ions.

28. The method according to claim 27, wherein the metal atoms and/or ions comprise at least one lanthanoid.

29. The method according to claim 28, wherein the at least one lanthanoid is selected from the group consisting of gadolinium, europium, terbium, and mixtures of at least two of gadolinium, europium, and terbium.

30. The method according to claim 24, wherein the single-wall carbon nanotubes have an average diameter of 2 nm and an average length of 1 μm.
